(19) United States
(12) Patent Application Publication
(10) Pub. No.: US 2019/0329039 A1
(43) Pub. Date: Oct. 31, 2019

(54) Anodic Stimulation in an Implantable Stimulator System Using Asymmetric Anodic and Cathodic Stimulation Pulses

(71) Applicant: Boston Scientific Neuromodulation Corporation, Valencia, CA (US)

(72) Inventors: Goran N. Marnfeldt, Valencia, CA (US); Michael A. Moffitt, Saugus, CA (US); Stephen Carcieri, Los Angeles, CA (US)

(21) Appl. No.: 16/373,448

(22) Filed: Apr. 2, 2019

Related U.S. Application Data
(60) Provisional application No. 62/663,794, filed on Apr. 27, 2018.

(51) Int. Cl. A61N 1/36

(52) U.S. Cl. CPC A61N 1/36103; A61N 1/0534; A61N 1/36196; A61N 1/36192

(57) ABSTRACT

Recognizing that anodic stimulation may require higher amplitudes or charge than cathodic stimulation in some tissues, new pulsing waveforms for a stimulator device, and particularly useful during monopolar stimulation, are described employing therapeutically-effective anodic and cathodic stimulation pulses at the lead-based electrode(s). The pulses are monophasic, with the amplitude or charge of the anodic monophasic pulses being higher than the cathodic monophasic pulses. To provide charge balance at each electrode, a pulse packet may be defined having a plurality of cathodic monophasic pulses and perhaps only a single anodic monophasic pulse. Because the polarity of cathodic monophasic pulses in each packet may charge balance with the anodic monophasic pulse(s), active charge recovery such as by the use of biphasic pulses may not be necessary, although passive charge recovery can be used if desired.

### CROSS REFERENCE TO RELATED APPLICATIONS

This is a non-provisional application of U.S. Provisional Patent Application Ser. No. 62/663,794, filed Apr. 27, 2018, to which priority is claimed, and which is incorporated herein by reference in its entirety.

### FIELD OF THE INVENTION

This application relates to Implantable Pulse Generators (IPGs), and more specifically to circuitry and methods for using anodic stimulation using asymmetric stimulation pulses.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) or Spinal Cord Stimulation (SCS) system, such as that disclosed in U.S. Pat. No. 6,516,227 and U.S. Patent Application Publication 2016/0184591. However, the present invention may find applicability with any implantable neurostimulator device system.

An SCS or DBS system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12 can also comprise an electrode (Ec).

In a SCS application, as is useful to alleviate chronic back pain for example, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In a DBS application, as is useful in the treatment of tremor in Parkinson's disease for example, the IPG 10 is typically implanted under the patient's clavicle (collarbone). Percutaneous leads 15 are tunneled through the neck and the scalp where the electrodes 16 are implanted through holes drilled in the skull and positioned for example in the subthalamic nucleus (STN) and the pedunculopontine nucleus (PPN) in each brain hemisphere. In other IPG examples designed for implantation directly at a site requiring stimulation, the IPG can be lead-less, having electrodes 16 instead appearing on the body of the IPG 10. The IPG lead(s) can be integrated with and permanently connected to the IPG 10 in other solutions.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases such as 30a and 30b, as shown in the example of FIG. 2A. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b ; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I from the tissue. Note that at any time the current sunk from the tissue (e.g., −I at E1 during phase 30a) equals the current sourced to the tissue (e.g., +I at Ec during phase 30a) to ensure that the net current injected into the tissue is zero.

IPG 10 as mentioned includes stimulation circuitry 28 to form prescribed stimulation at a patient's tissue. FIG. 3 shows an example of stimulation circuitry 28, which includes one or more current sources 40, and one or more current sinks 42ᵢ. The sources and sinks 40ᵢ and 42ᵢ can comprise Digital-to-Analog converters (DACs), and may be referred to as PDACs 40_{I} and NDACs 42_{I} in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40, and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus PDAC 40_{C} and NDAC 42₁ are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ei 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. patent application Ser. No. 15/695,965, filed Sep. 5, 2017, and U.S. Provisional Patent Application Ser. No. 62/559,247, filed Sep. 15, 2017.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blockings capacitors C1 and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors C1 and Cc is actively recovered, and thus voltages Vc1 and Vcc increase and return to 0V at the end the second pulse phase 30b.

To recover all charge by the end of the second pulse phase 30b of each pulse (Vc1=Vcc=0V), the first and second phases 30a and 30b are charged balanced at each electrode, with the first pulse phase 30a providing a charge of −Q (−I*PW) and the second pulse phase 30b providing a charge of +Q (+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balance if the product of the amplitude and pulse widths of the two phases 30a and 30b are equal, or if the area under each of the phases is equal, as is known.

FIG. 3 shows that stimulation circuitry 28 can include passive recovery switches 41ᵢ, which are described further in U.S. Patent Application Publications 2018/0071527 and 2018/0140831. Passive recovery switches 41ᵢ may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially-decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

FIG. 4 shows an external trial stimulation environment that may precede implantation of an IPG 10 in a patient, particularly in an SCS application. During external trial stimulation, stimulation can be tried on a prospective implant patient without going so far as to implant the IPG 10. Instead, one or more trial electrode arrays 17′ (e.g., one or more trial percutaneous leads 15 or trial paddle leads 19) are implanted in the patient's tissue at a target location 52, such as within the spinal column as explained earlier. The proximal ends of the trial electrode array(s) 17′ exit an incision 54 in the patient's tissue and are connected to an External Trial Stimulator (ETS) 50. The ETS 50 generally mimics operation of the IPG 10, and thus can provide stimulation to the patient's tissue as explained above. See, e.g., U.S. Pat. No. 9,259,574, disclosing a design for an ETS. The ETS 50 is generally worn externally by the patient for a short while (e.g., two weeks), which allows the patient and his clinician to experiment with different stimulation parameters to hopefully find a stimulation program that alleviates the patient's symptoms (e.g., pain). If external trial stimulation proves successful, the trial electrode array(s) 17′ are explanted, and a full IPG 10 and a permanent electrode array 17 (e.g., one or more percutaneous 15 or paddle 19 leads) are implanted as described above; if unsuccessful, the trial electrode array(s) 17′ are simply explanted.

Like the IPG 10, the ETS 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power.

FIG. 5 shows various external devices that can wirelessly communicate data with the IPG 10 or ETS 50, including a patient, hand-held external controller 60, and a clinician programmer 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example, and may comprise a controller dedicated to work with the IPG 10 or ETS 50. External controller 60 may also comprise a general purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-filed magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 82 provided on the display 74 of the computing device 72. As one skilled in the art understands, the GUI 82 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 82, can also enable communications via antennas 80a or 80b to communicate stimulation parameters chosen through the GUI 82 to the patient's IPG 10.

The user interface of the external controller 60 may provide similar functionality because the external controller 60 can include the same hardware and software programming as the clinician programmer. For example, the external controller 60 includes control circuitry 66 similar to the control circuitry 88 in the clinician programmer 70, and may similarly be programmed with external controller software stored in device memory.

### SUMMARY

A stimulator device, which may comprise: a plurality of electrode nodes, each electrode node configured to be coupled to one of a plurality of electrodes configured to contact a patient's tissue; and stimulation circuitry configured to provide a repeating packet of pulses at at least two of the electrode nodes selected to create a stimulation current through the patient's tissue, wherein the pulses in each packet at at least one of the at least two electrode nodes comprise at least one monophasic anodic pulse and a plurality of monophasic cathodic pulses, wherein the at least one monophasic anodic pulse comprises a first amplitude or charge, and wherein the plurality of monophasic cathodic pulses each comprise an amplitude or charge less than the first amplitude or charge.

The at least one monophasic anodic pulse and the plurality of monophasic cathodic pulses in each packet may be separated by gaps during which the stimulation current is not created through the patient's tissue. The amplitudes or charges of the plurality of monophasic cathodic pulses may be equal in each packet. Each packet may comprises only the at least one monophasic anodic pulse and the plurality of monophasic cathodic pulses.

The at least one monophasic anodic pulse and the plurality of monophasic cathodic pulses in each packet may or may not be charge balanced at each of the at least one electrodes. The pulses in each of the packets may comprise a single monophasic anodic pulse and a plurality of monophasic cathodic pulses. Each packet may further comprise a passive charge recovery phase, wherein during the passive charge recovery phase the stimulation circuitry is configured to short the at least two of the electrode nodes to a reference voltage.

The first amplitude or charge of the at least one monophasic anodic pulse and each amplitude or charge of the plurality of monophasic cathodic pulses may be selected to be therapeutically effective to recruit neural elements in the patient's tissue. Alternatively, the first amplitude or charge of the at least one monophasic anodic pulse may be selected to be therapeutically effective to recruit neural elements in the patient's tissue, but each amplitude or charge of the plurality of monophasic cathodic pulses may be selected to not be therapeutically effective to recruit neural elements in the patient's tissue.

The at least one monophasic anodic pulse may comprise a passive monophasic anodic pulse, wherein the stimulation circuitry is configured to provide the passive monophasic anodic pulse by shorting the at least two of the electrode nodes to a reference voltage. The stimulation circuitry may be configured to provide the at least one monophasic anodic pulse and the plurality of monophasic cathodic pulses at a constant frequency at each of the at least two of the electrode nodes.

The stimulator device may further comprise a case for housing the stimulation circuitry, wherein the case is conductive, and wherein the conductive case comprises one of the plurality of electrodes. One of the at least two of the electrode nodes may comprise an electrode node coupled to the conductive case. The stimulator device may further comprise at least one implantable lead wherein some of the electrodes are located on the at least one implantable lead. The at least one of the at least two electrode nodes may comprise an electrode node coupled to an electrode located on the at least one implantable lead.

Each electrode node may be coupled to an electrode through a DC-blocking capacitor. The first amplitude of the at least one monophasic anodic pulse and each amplitude of the plurality of monophasic cathodic pulses may comprise constant current amplitudes.

The stimulator device may comprise an implantable pulse generator, such as a deep brain stimulator. The stimulator device may also comprise an external stimulator.

The pulses in each packet at at least one other of the at least two electrode nodes may comprise at least one monophasic cathodic pulse corresponding in time to the at least one monophasic anodic pulse and a plurality of monophasic anodic pulses each corresponding in time to one of the plurality of monophasic cathodic pulses, wherein the at least one monophasic cathodic pulse comprises the first amplitude or charge, and wherein each of the plurality of monophasic anodic pulses comprises the amplitude or charge of its corresponding monophasic cathodic pulse.

The at least one monophasic anodic pulse and the plurality of monophasic cathodic pulses may be charge imbalanced at each of the at least one electrodes in a plurality of the packets, but the charge may be balanced at each of the at least one electrodes over a duration of the plurality of the packets.

### BRIEF DESCRIPTION OF THE DRAWINGS

FIG. 1 shows an Implantable Pulse Generator (IPG), in accordance with the prior art.

FIGS. 2A and 2B show an example of stimulation pulses (waveforms) producible by the IPG or by an External Trial Stimulator (ETS), in accordance with the prior art.

FIG. 3 shows an example of stimulation circuitry useable in the IPG or ETS, in accordance with the prior art.

FIG. 4 shows an ETS environment useable to provide stimulation before implantation of an IPG, in accordance with the prior art.

FIG. 5 shows various external devices capable of communicating with and programming stimulation in an IPG or ETS, in accordance with the prior art.

FIGS. 6 shows biphasic waveforms producible by an IPG or ETS having an anodic first pulse phase at a lead-based electrode and useable during monopolar stimulation using the case electrode as a return.

FIG. 7A shows an example of waveforms producible by an IPG or ETS having a repeating packet of pulses, with each packet including a high amplitude monophasic anodic pulse followed by a plurality of lower amplitude monophasic cathodic pulses, where the pulses are charge balanced at each electrode.

FIGS. 7B and 7C show other examples of waveforms similar to FIG. 7A in which the anodic and cathodic pulses are ordered differently inside each packet.

FIG. 8 shows another example of waveforms in which the cathodic pulses have the same charge but are shaped differently by varying their amplitudes or pulse widths.

FIG. 9 shows another example of waveforms in which the cathodic pulses have differing amounts of charge.

FIG. 10 shows another example of waveforms having more than one anodic pulse in addition to the cathodic pulses.

FIG. 11 shows another example of waveforms in which two lead-based electrodes are selected along with the case electrode during monophasic stimulation.

FIG. 12 shows another example of waveforms in which the anodic pulse is passively formed using passive recovery circuitry in the IPG or ETS.

FIG. 13 shows another example of waveforms in which the anodic and cathodic pulses are formed at two lead-based electrodes using bipolar stimulation.

FIG. 14 shows another example of waveforms in which only the anodic pulse is therapeutically effective, and in which the cathodic pulses are not therapeutically effective but are instead used for charge recovery.

FIG. 15 shows another example of waveforms in which the anodic and cathodic pulses do not occur at a constant frequency.

FIG. 16 shows another example of waveforms with each packet including a high amplitude monophasic cathodic pulse followed by a plurality of lower amplitude monophasic anodic pulses, where the pulses are charge balanced at each electrode.

FIG. 17 shows another example of waveforms in which the anodic and cathodic pulses are charge imbalanced at each electrode in each packet.

FIG. 18 shows another example of waveforms in which the anodic and cathodic pulses are charge imbalanced at each electrode in each packet, but where different packets are in sum charge balanced at each electrode.

FIGS. 19A and 19B show other examples of waveforms in which charge is imbalanced within the pulse packets to affect a DC current bias in the tissue.

FIG. 20 shows a Graphical User Interface (GUI) rendered on an external device and useable to program an IPG or ETS with any of the waveforms previously illustrated.

FIG. 21 shows circuitry in the IPG or ETS for receiving stimulation parameters as provided by the GUI of the external device and to provide the specified waveforms.

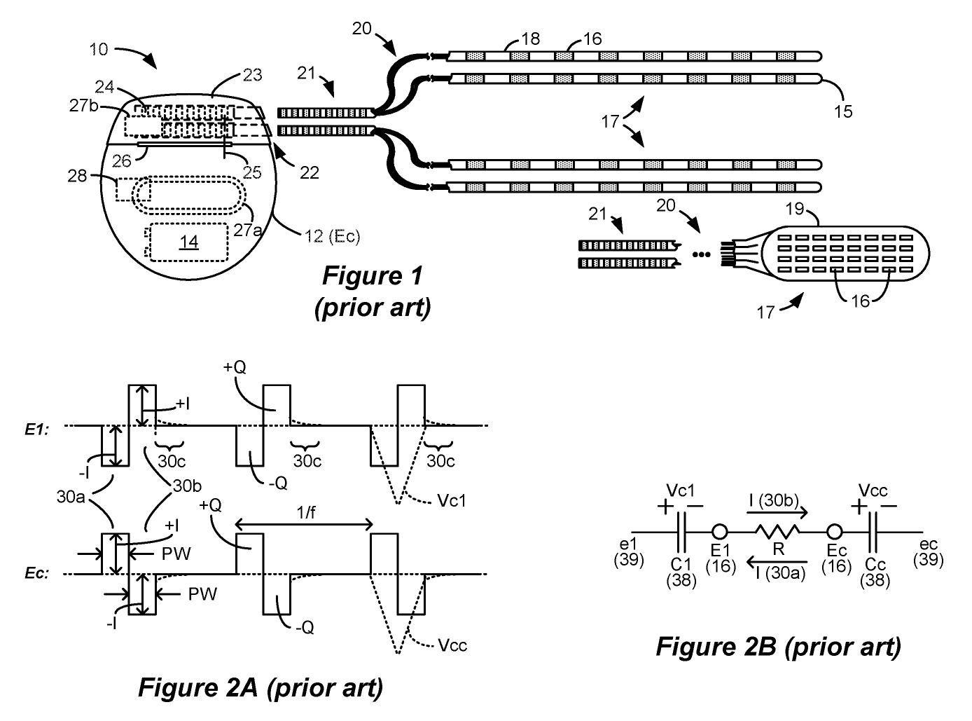

### DETAILED DESCRIPTION

Especially in the DBS context, monopolar stimulation is traditionally performed using cathodic stimulation—that is, by using pulses at the lead-based electrodes (e.g., E1) whose first (or only) pulse phase 30a is cathodic (negative). As a result, and to ensure that the net charge injection into the tissue at any time is zero, the corresponding first (or only) pulse phase 30a at the case electrode would be anodic (positive). Such traditional cathodic monopolar stimulation was shown in FIG. 2A via the use of biphasic pulses, although as described earlier, a single pulse phase followed by passive recovery may be used as well.

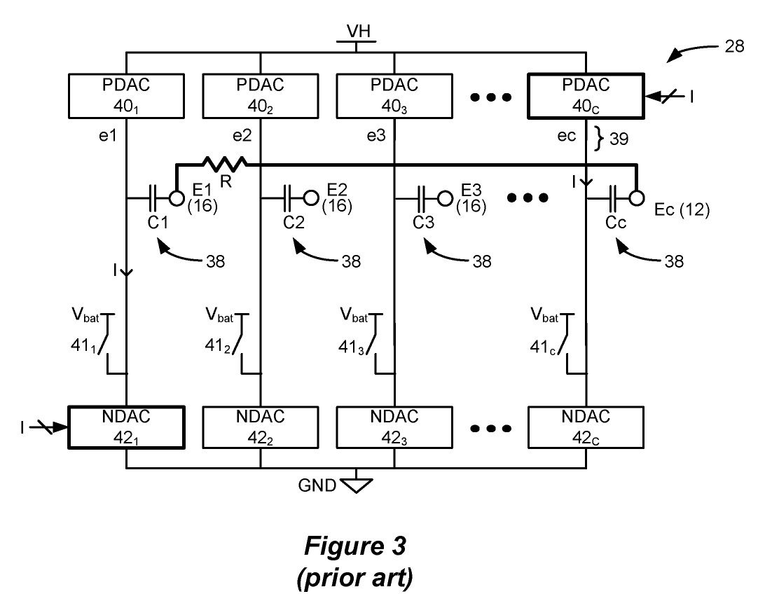

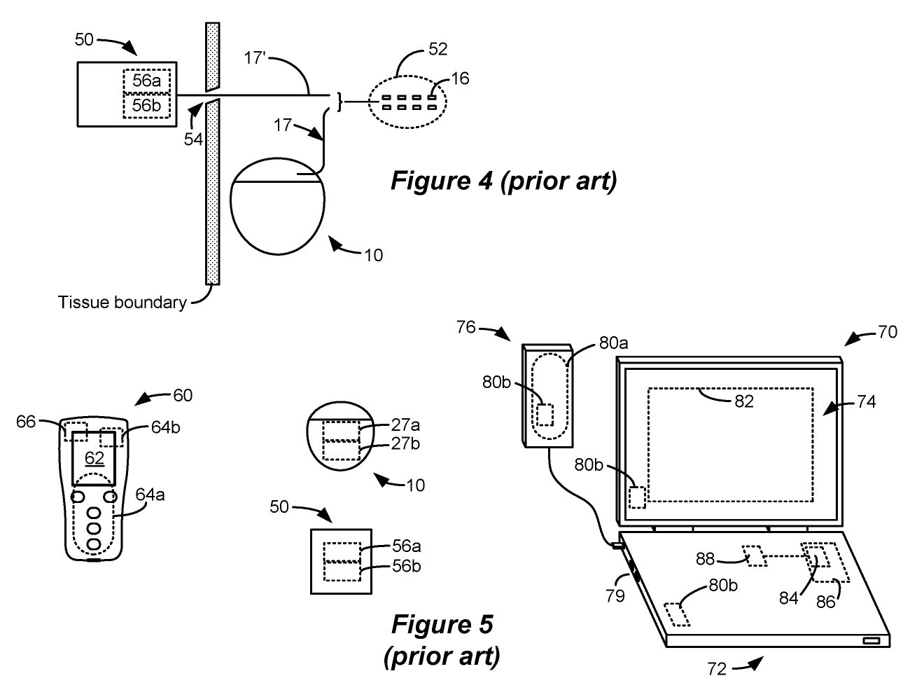

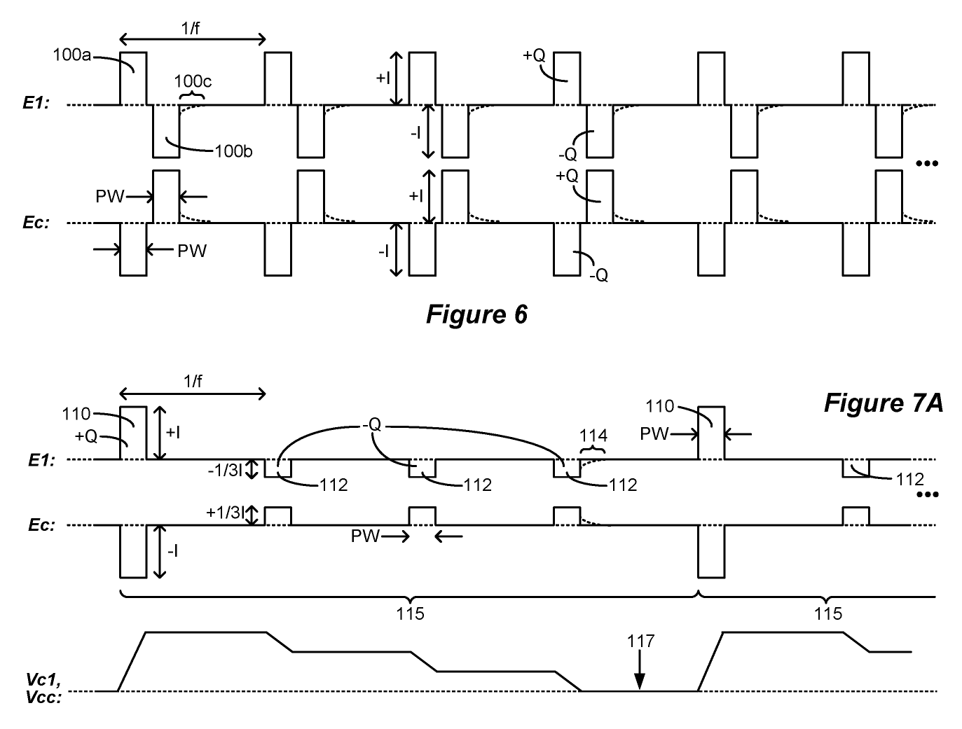

However, anodic stimulation—that is, using pulses at the lead-based electrodes whose first (or only) pulse phase 30a is anodic (positive)—may provide superior results. Anodic monopolar stimulation is shown in FIG. 6, again using charge-balanced biphasic pulses. As shown, a lead-based electrode has been selected (e.g., E1, although any other electrode 16 could be chosen) to provide stimulation along with the case electrode Ec. To ensure that there is no net charge injection into the tissue at any given time, the case electrode Ec is provided with pulse phases of opposite polarity, i.e., with a cathodic first pulse phase 100a followed by a charge-balanced anodic second pulse phase 100b. As described earlier, the second pulse phases 100b can be followed by a passive recovery phase 100c, during which the passive recovery switches 41ᵢ (FIG. 3) can be closed to equilibrate any remaining charge on capacitances in the current path.

While either a cathodic pulse (FIG. 2A) or an anodic pulse (FIG. 6) may be effective to provide stimulation to the tissue, the amplitude of the current needed may vary. For example, to stimulate the same tissue, the amplitude of an anodic current may need to be 1.5 to 3 times higher than a cathodic current. The charge of an anodic pulse may also need to be higher than cathodic pulses to stimulate the same tissue.

This may make the use of anodic stimulation as depicted in FIG. 6 difficult. Assume for example that effective anodic stimulation for a given patient requires an anodic current of amplitude +I at electrode E1 during the first phase 100a. This could render the current used during active charge recovery—i.e., during the second pulse phase 100b—too high. If the second pulse phase 100b is assumed to have the same pulse width (PW) as the first pulse phase 100a, charge balanced at E1 would mandate that the second pulse phase 100b would also have the same amplitude as the first pulse phase (although of opposite polarity, −I). A cathodic current of this amplitude could be 1.5 to 3 times higher than needed to stimulate the tissue. Thus, even though the second pulse phase 100b is intended merely as a means of charge recovery and not as a therapeutically-significant pulse, the cathodic second pulse phase 100b runs the risk of over-stimulating the tissue. In fact, the cathodic current in the second pulse phase could be painful to the patient or create other unwanted side effects.

Recognizing that anodic stimulation may require higher amplitudes or charge than cathodic stimulation, new pulsing waveforms particularly useful during monopolar stimulation are described employing therapeutically-effective anodic and cathodic stimulation pulses at the lead-based electrode(s). In some examples, the pulses are monophasic, with the amplitude or charge of the anodic monophasic pulses being higher than the cathodic monophasic pulses. To provide charge balance at each electrode, a pulse packet may be defined having a plurality of cathodic monophasic pulses and perhaps only a single anodic monophasic pulse. Because the polarity of cathodic monophasic pulses in each packet may charge balance with the anodic monophasic pulse(s), active charge recovery such as by the use of biphasic pulses may not be necessary, although passive charge recovery can be used. Use of both anodic and cathodic currents can be beneficial because such currents may recruit different neural elements in the patient's tissue (e.g., cells versus fibers).

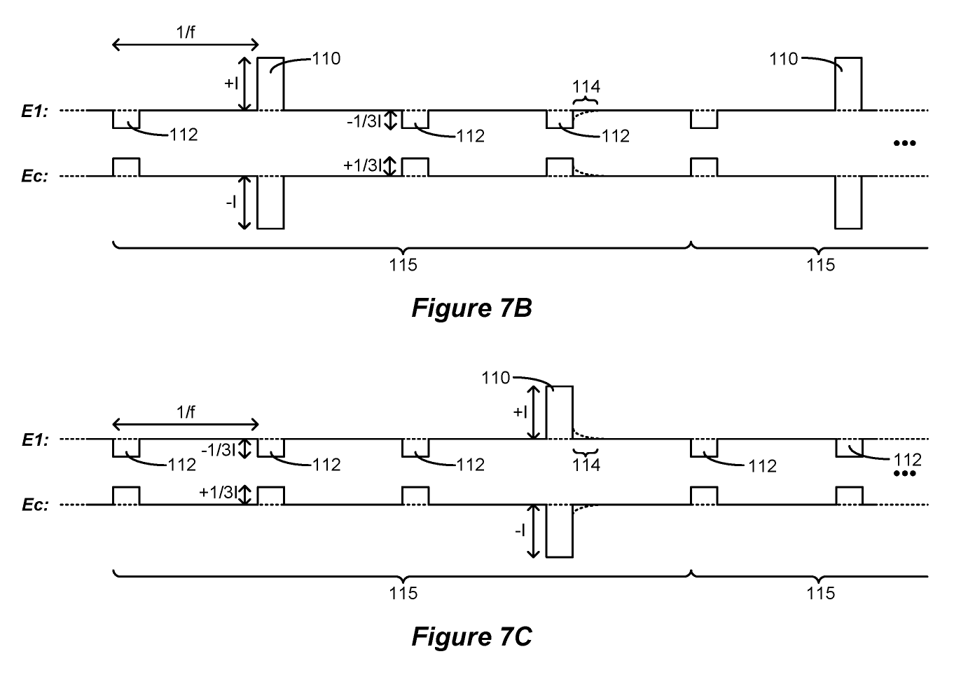

A first example of new pulsing waveforms as just described is shown in FIG. 7A. The pulses in FIG. 7A are monopolar, with one or more lead-based electrodes 16 (e.g., E1) and the case electrode 12 Ec selected to provide monopolar stimulation. In this example, monophasic anodic stimulation pulses 110 and monophasic cathodic pulses 112 are issued at a frequency f, which frequency has presumably been found to be effective for the patient. The pulses are organized in packets 115, and in the example shown each packet 115 comprises at the lead-based electrode E1 a single monophasic anodic stimulation pulse 110 followed by a plurality (e.g., three) of monophasic cathodic stimulation pulses 110. To prevent net charge injection into the tissue at any given time, the pulse packet 115 at the case electrode Ec comprises pulses 110 and 112 of the same amplitudes but opposite polarities: a single monophasic cathodic stimulation pulse 110 followed by a plurality (e.g., three) of monophasic anodic stimulation pulses 112. While the opposite-polarity return electrode Ec is shown in subsequent figures for completeness, discussion in this disclosure will focus on the pulses formed at the lead-based electrodes (e.g., E1) involved in neural recruitment.

The pulse packets 115 repeat as shown, such that the pulses 110 or 112 issued at the specified frequency, f. Preferably, the pulses 110 and 112 in each packet 115 are separated by gaps during which a stimulation current is not created through the patient's tissue.

As discussed above, stimulating tissue at a lead-based electrode E1 may require a higher-amplitude anodic current and a lower-amplitude cathodic current, and this is reflected in the amplitudes of the anodic stimulation pulses 110 and 112. In this example, while the amplitude of the anodic stimulation pulses 110 at E1 are +I, the amplitude of the cathodic stimulation pulses 112 are −1/3I. If an effective anodic current is assumed to be three times the amplitude of a cathodic current, these pulses 112 and 110 should each be therapeutically effective to recruit the tissue proximate to electrode E1. Beneficially, the anodic and cathodic currents may recruit different neural elements in the tissue.

Beneficially, each of the pulses 110 and 112 comprises a single phase (i.e., are monophasic) and do not require second (opposite) pulses phases for the purpose of active charge recovery. This simplifies the issuance of the pulses, and reduces the amount of power needed to form the pulses compared to techniques like FIG. 6A that use biphasic pulses. Nonetheless, charge recovery at each electrode can still occur. This is because each of the cathodic pulses 112 in a packet 115 may be structured to recover charge built up by the single anodic pulse 110 over the duration of that packet. In other words, the cathodic pulses 112 are structured to provide a total summed charge of −Q while the anodic pulse 110 is structured to provide a total charge of +Q in each packet.

Charge balancing can be achieved in different ways, but in FIG. 7A is achieved by inversely scaling the amplitude of the cathodic pulses 112 in accordance with the number of those pulses 112 included in each packet 115. Assuming pulses 110 and 112 all have the same pulse width (PW), and that three cathodic pulses 112 are used, the amplitude of the cathodic pulses are set to −1/3I (−1/3I) the amplitude of the anodic pulse 110 (+I). The charge of each cathodic pulse 112 is thus equal to −1/3*I*PW, and thus in sum equals −Q=−I*PW, which is equal and opposite to the charge +Q=+I*PW of the anodic pulse 110. In other examples also achieving charge balance at each electrode, two cathodic pulses 112 may be used in each packet 115 with amplitudes −1/2I, or four cathodic pulses 112 may be used with amplitudes −1/4I, etc.

FIG. 7A shows how the pulses 110 and 112 achieve charge recovery by review of the voltages that build up on capacitances in the current path, such as on the DC-blocking capacitors 38 (C1 and Cc) associated with electrode E1 and the case electrode Ec. Charge will build up during the anodic pulse 110, and Vc1 and Vcc across C1 and Cc (FIG. 2B) will rise. This charge is recovered during each subsequent cathodic pulse 112, and eventually Vc1 and Vcc fall back to zero (117) at the end of the packet 115. In other words, by charge balancing the sum of the charge of the cathodic pulses 112 to equal the opposite charge of the anodic pulse 112, complete charge recovery is achieved by the end of the pulse packet 115.

Passive charge recovery may also be used to ensure complete charge recovery, because as noted earlier intentional charge balancing at a given electrode may not be perfect given non-idealities. In this regard, passive charge recovery periods 114 are included after the last pulse in each packet 115. By way of review, passive charge recovery occurs by closing at least the passive recovery switches 41₁ and 41_{c} (FIG. 3) associated with the electrodes E1 and Ec chosen for stimulation, which couples electrode nodes e1 and ec 39 to a common reference voltage (Vbat). All passive recovery switches 41ᵢ may also be closed during periods 114 if desired.

FIGS. 7B and 7C show that the order of the anodic pulse 110 and cathodic pulses 112 in each packet 115 does not matter and can be modified. Thus, in FIG. 7B, a cathodic pulse 112 occurs first at E1 in each packet 115, followed by the anodic pulse 110, and followed by two cathodic pulses 112. In FIG. 7C, the anodic pulse 110 occurs at the end of the packet 115 after all three cathodic pulses 112. This still arrives at a charge-balanced solution at each electrode, and again passive charge recovery 114 can be used after each last pulse in the packet 115. In fact, and recognizing that the packets 115 repeat and that the order of the pulses 110 and 112 in each packet can vary, passive charge recovery 114 can actually occur at any set position in the packet 115—after the first pulse, after the second pulse, etc. Further, passive charge recovery 114 can also occur after every pulse 110 and 112 if desired.

FIG. 8 shows that charge balancing can also be achieved by varying the pulse widths of the pulses in the packet 115. Three cathodic pulses 112 are again shown, with each having different shapes. The first has an amplitude of −1/6I and a pulse width 2PW; the second has an amplitude −2/3I and a pulse width of 1/2PW; and the third has an amplitude of −1/3I and a pulse width PW. The charge of each cathodic pulse 112 is −1/3*I*PW, and so all three in sum have a charge −Q=−I*PW, equal and opposite to the charge of the anodic pulse 110, +Q=+I *PW. Although not shown, note that the pulses 110 and 112 do not need to be square pulses of constant current amplitudes, but could have random shapes, while still being charge balanced.

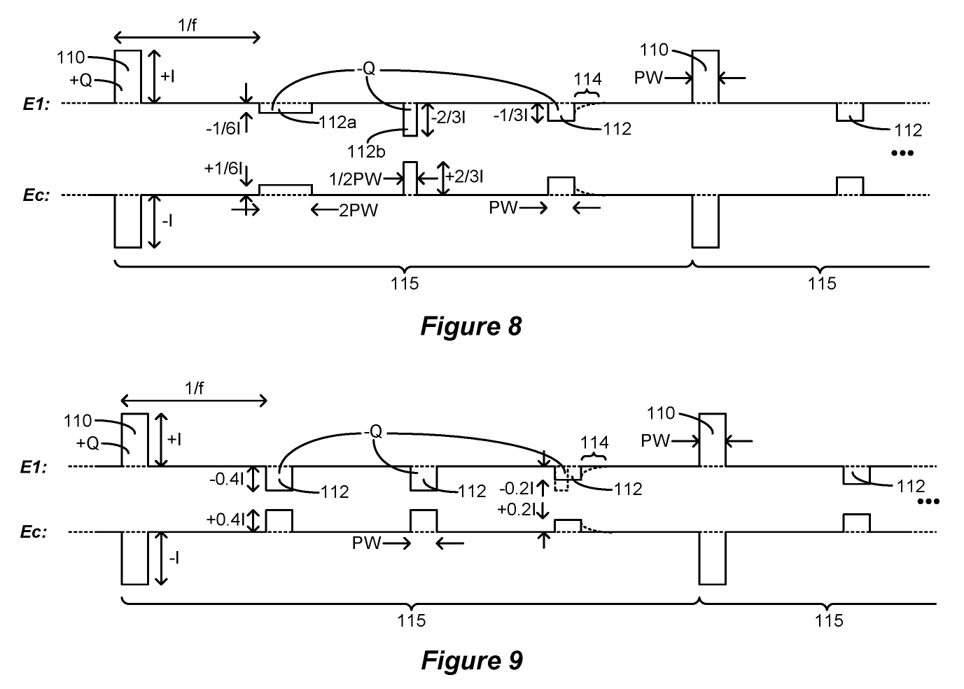

FIG. 9 shows that charge balancing can be achieved at each electrode even if the pulses have different amounts of charge. As shown, the first two cathodic pulses 112 have an amplitude of −0.4I, while the last cathodic pulse 112 has an amplitude of −0.2I. The total charge of the cathodic pulses 112 is again −Q=−I*PW. While this is charge balanced with the anodic pulse 110, note that the cathodic pulses 112 may not all have the same therapeutic efficacy. For example, assume that it is known in a particular tissue that an effective anodic current is 2.5 times an effective cathodic current. The cathodic pulses 112 at −0.4I would then have the same efficacy as the anodic pulse 110 at +I (i.e., 1/0.4=2.5). The last cathodic pulse 112 with amplitude −0.21 may be less effective, but this can be tolerable to arrive at a charge balanced solution. Alternatively, and as shown in dotted lines, the last cathodic pulse 112 can also have the same amplitude of −0.4I as the other pulses 112, and hence may be as therapeutically effective, but ½ the pulse width to also arrive at a charge balanced solution.

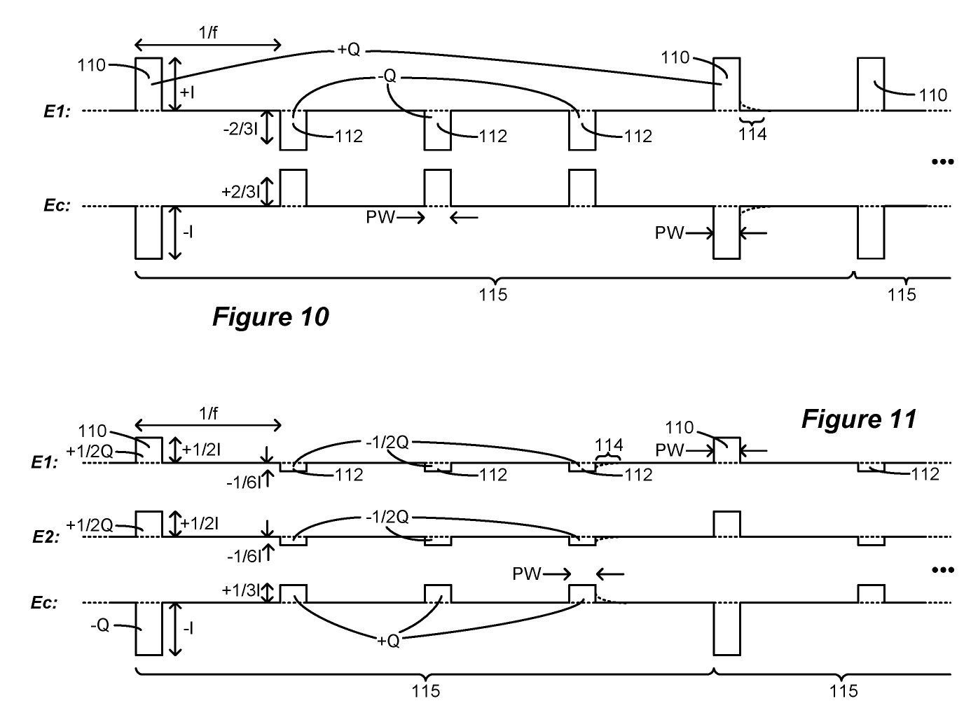

It is not strictly necessary that each pulse packet 115 have only one anodic pulse 110. Assume for example that an effective anodic current is 1.5 times an effective cathodic current. In FIG. 10, a pulse packet 115 thus includes two anodic pulses 110 of amplitude +I, and three cathodic pulses 112 of amplitude −2/3I. These pulses 110 and 112 should have the same therapeutic effectiveness (i.e., 1/[2/3]=1.5). Assuming in this example that the pulses widths of pulses 110 and 112 are the same, the sum of the charge of the anodic pulses 110 (2*+I*PW) is equal and opposite to the sum of the charge of the cathodic pulses 112 (3*−2/3I*PW), and so the pulses 110 and 112 are charge balanced within each packet 115. Again, the order of the anodic pulses 110 and the cathodic pulses 112 within a packet 115 can be modified, and the order need not be the same in each of the packets 115.

FIG. 11 shows applicability of the disclosed technique when more than one lead-based electrode 16 is selected for monopolar stimulation along with the case electrode Ec. This can be useful because selection of more than one lead-based electrode allows for the formation of a virtual electrode or virtual pole between the two selected electrodes, as is known. See, e.g., U.S. Provisional Patent Application Ser. No. 62/598,114, filed Dec. 13, 2017.

In this example, two electrodes (e.g., E1 and E2) are selected in addition to the case electrode Ec. Each electrode E1 and E2 receives of the same current: +1/2I during anodic pulses 110, and −1/6I during cathodic pulses 112. Note however that electrodes E1 and E2 do not need to share current equally: for example, E1 may receive 75% of the current (forming pulses 110 and 112 with amplitudes of +3/4I and −1/4I), with E2 receiving the remaining 25% (forming pulses 110 and 112 with amplitudes of +1/4I and −1/12I). The total current from E1 and E2 equals the opposite current at Ec at any point in time, ensuring no net charge injection into the tissue. Further, the charge at each electrode E1 and E2 is charge balanced between the anodic pulse 110 (+1/2Q) and the cathodic pulses 112 (−1/2Q).

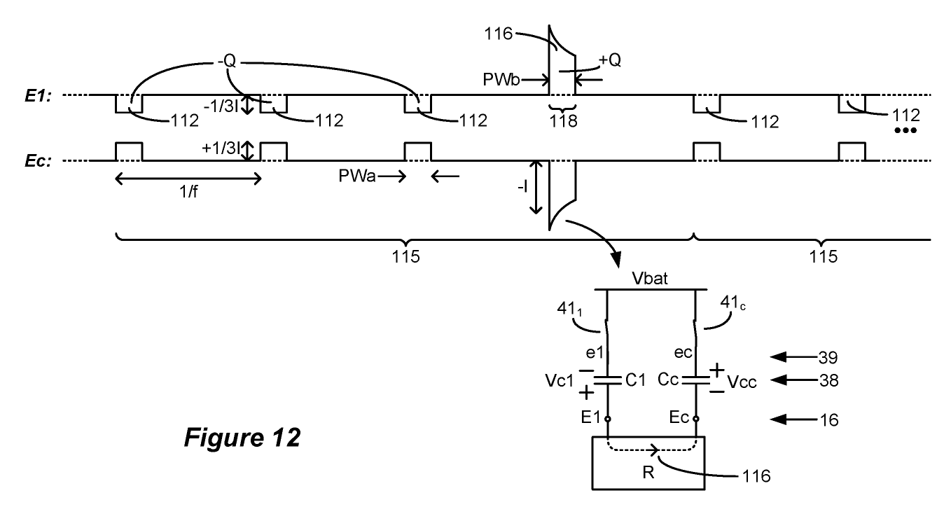

FIG. 12 provides a different example in which anodic pulses 116 are not actively driven (e.g., by the DACs in stimulation circuitry 28; FIG. 3), but are instead passively formed using passive recovery switches 41ᵢ. Cathodic pulses 112 are actively driven (28; FIG. 3) as before, followed by the passive anodic pulse 116 at the end of the packet 115. The circuit diagram in FIG. 12 illustrates generation of the passive anodic pulse 116. After the cathodic pulses 112, capacitors C1 and Cc 38 coupled to the electrodes E1 and Ec would be charged (Vc1, Vcc) with the polarities as shown. When the passive recovery switches 41₁ and 41_{c} are closed during the passive anodic pulse (118), thus shorting electrode nodes e1 and ec 39 to the Vbat reference voltage, equilibration of the charge on these capacitors will cause a current flow from E1 to Ec through the patient's tissue, R. Given the R-C nature of this circuit, the current during the passive anodic pulse 116 will exponentially decay. Note that the pulse widths of the cathodic pulses 112 and the passive anodic pulse 116 can be different (e.g., PWa and PWb). In particular, it may be useful that the pulse width PWb of the passive anodic pulse 116 (i.e., the duration of 118) be long enough to allow the charge across the capacitors to exponentially decay back to 0 Volts. However, this is not strictly necessary, as the charge of the cathodic pulse 112 can be modified to, in sum, equal the charge dissipated during the passive anodic pulse 116 so that charge is once again balanced, with the sum of the charge of the cathodic pulses 112 (−Q) equaling the opposite charge of the passive anodic pulse 116 (+Q).

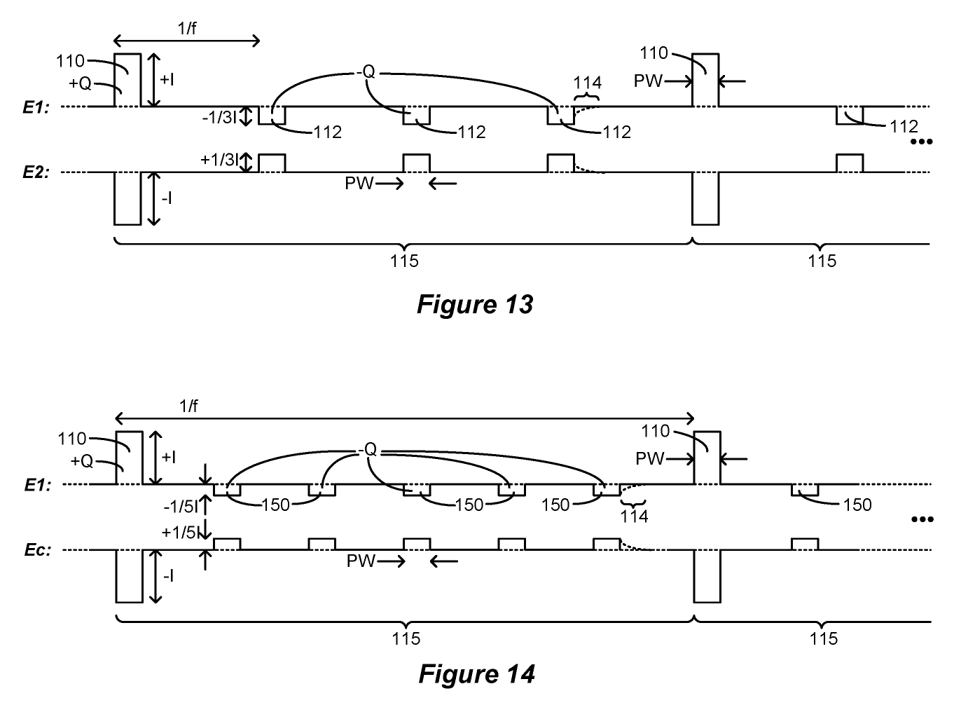

To this point in the application, it has been assumed that stimulation occurs using monopolar stimulation, with one of the selected electrodes comprising the conductive case electrode Ec. However, this is not strictly necessary. FIG. 13 provides an example using bipolar stimulation in which two lead-based electrodes 16 are selected for stimulation, such as E1 and E2.

Although not illustrated, FIG. 13 further suggests how monopolar stimulation can occur without use of the case electrode Ec. As FIG. 11 illustrated earlier, a current can be shared between two electrodes 16—e.g., E1 and E2. In FIG. 13, pseudo-monopolar simulation can thus be created by sharing a return current (normally flowing to Ec) between the remaining electrodes E2-E32. Sharing the current at electrodes E2-E32 provides in sum a large-area return, akin to the case electrode Ec. In other words, E1 as a selected active electrode can provide pulses 110 and 112 as shown, with E2-E32 sharing the return current. Further, the return current can be shared between E2-E32 and the case electrode Ec.

It is not strictly necessary that the anodic and cathodic pulses 110 and 112 all comprise therapeutically-significant pulses. Consider FIG. 14, which provides anodic pulses 110 with an anodic current of +I (110) and five cathodic pulses 150 with a cathodic current of −1/5I (150). In this circumstance, and depending on the neural tissue at issue, the cathodic current may be of too low an amplitude to recruit neural elements, and thus will have little or no therapeutic effect. Such cathodic pulses 150 can nonetheless still be provided for the purpose of charge balancing at each electrode. In effect, only the anodic pulses 110 are therapeutically effective, with the frequency f of therapy thus being dictated by the timing between the anodic pulses 110.

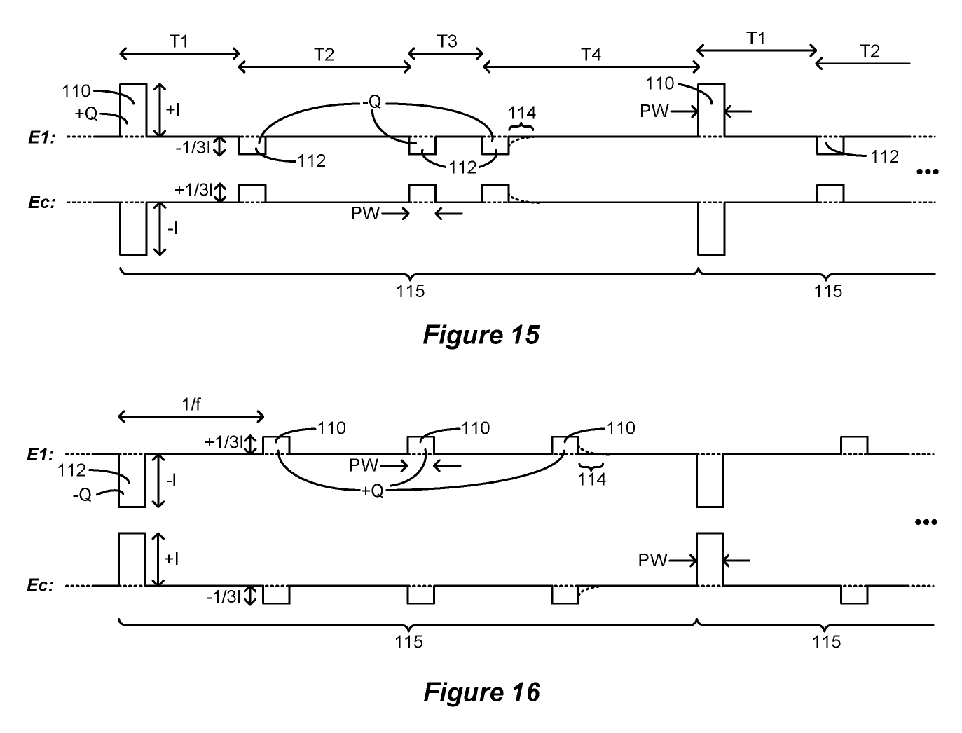

The anodic and cathodic pulses 110 and 112 do not have to occur at a constant frequency, f This is shown in FIG. 15, where the anodic pulse 110 and the first cathodic pulse 112 are separated by a time period T1; the first and second cathodic pulses 112 are separated by T2; the second and last cathodic pulses 112 are separated by T3; and the last cathodic pulse 112 is separated from the anodic pulse 112 in the next packet 115 by T4.

FIG. 16 provides another example in which the packets 115 comprise a single cathodic pulse 112 and a number of anodic pulses 110 at lead-based electrode E1. Pulses of this nature would be useful in tissues that require a higher cathodic current and a smaller anodic current to be therapeutically effective.

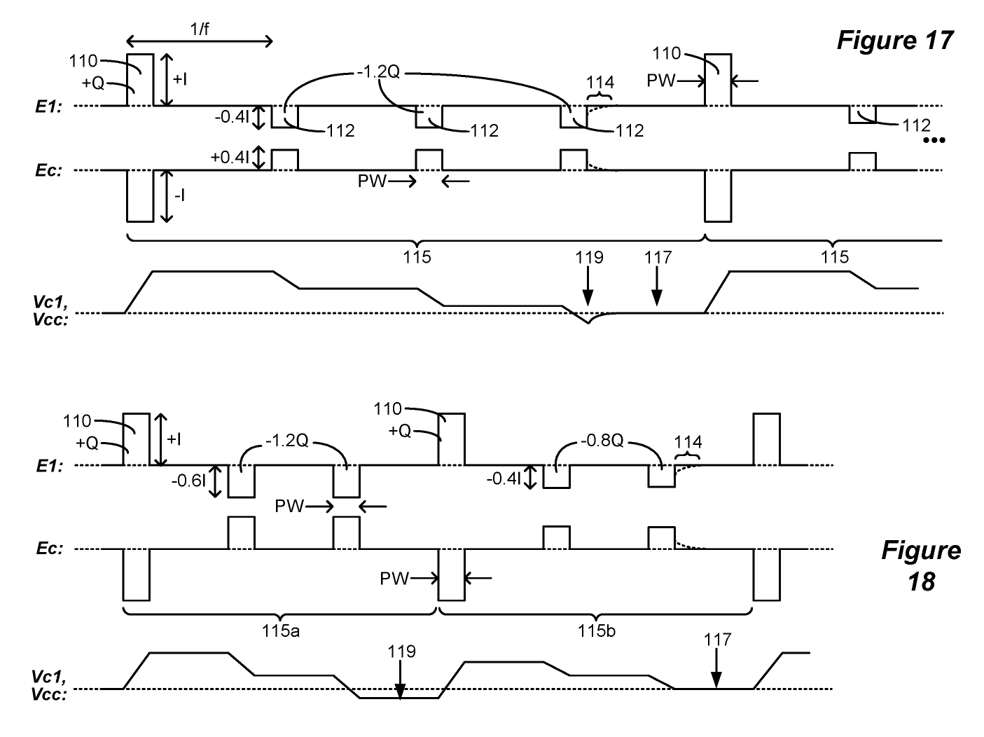

While beneficial that the anodic and cathodic pulses 110 and 112 be charge balanced at each electrode and in each packet 115, this is also not required in all useful implementations. For example, in FIG. 17, the cathodic currents of the cathodic pulses 112 have been changed to −0.4I. In this circumstance, the anodic pulse 110 (+Q) is not charge balanced with the cathodic pulses 112 (−1.2Q) in each packet 115, as evidenced by the remaining voltage Vc1 and Vcc (119) on DC-blocking capacitors C1 and Cc 38 (FIG. 3) after the issuance of the last cathodic pulse 112 in the packet 115. Nonetheless, this may be acceptable. First, it may be the case that an effective anodic current +I is 2.5 times an effective cathodic current, making −0.4I a logical choice for the cathodic current, even though this results in charge imbalanced pulses in each packet 115. Further, charge imbalance may be addressed in other ways. For example, the extra −0.2Q of charge may be passively recovered during periods 114 as discussed earlier, thus returning Vc1 and Vcc to zero (117) before the start of the next packet 115.

FIG. 18 shows how charge imbalanced packets 115 can be used to provide a charge-balanced solution at each electrode over the sum of a plurality of packets. Shown are two packets 115a and 115b. In both packets 115a and 115b, the anodic pulse 110 has a charge of +Q=+I*PW. In Packet 115a, there are two cathodic pulses 112, each having a pulse width of PW, and a cathodic current of −0.6I. Therefore, in sum, the two cathodic pulses 112 in packet 115a have a total charge of −1.2Q, and thus packet 115a is charge imbalanced at electrode E1 (with a net charge of −0.2Q). Such charge imbalance is evidenced by the residual voltage Vc1 and Vcc (119) remaining on the DC-blocking capacitors C1 and Cc after the last cathodic pulse 112 in packet 115a. In packet 115b, there are again two cathodic pulses 112, each having a pulse width of PW, but a cathodic current of −0.4I. Therefore, in sum, the two cathodic pulses 112 in packet 115b have a total charge of −0.8Q, and thus packet 115b is charge imbalanced at electrode E1 (with a net charge of +0.2Q). Nonetheless, over the span of packets 115a and 115b, the pulses 110 and 112 are balanced at each electrode. Such charge balance between the packets 115a and 115b is evidenced by the fact that the voltage Vc1 and Vcc (117) remaining on the DC-blocking capacitors C1 and Cc after the last cathodic pulse 112 in packet 115b is zero.

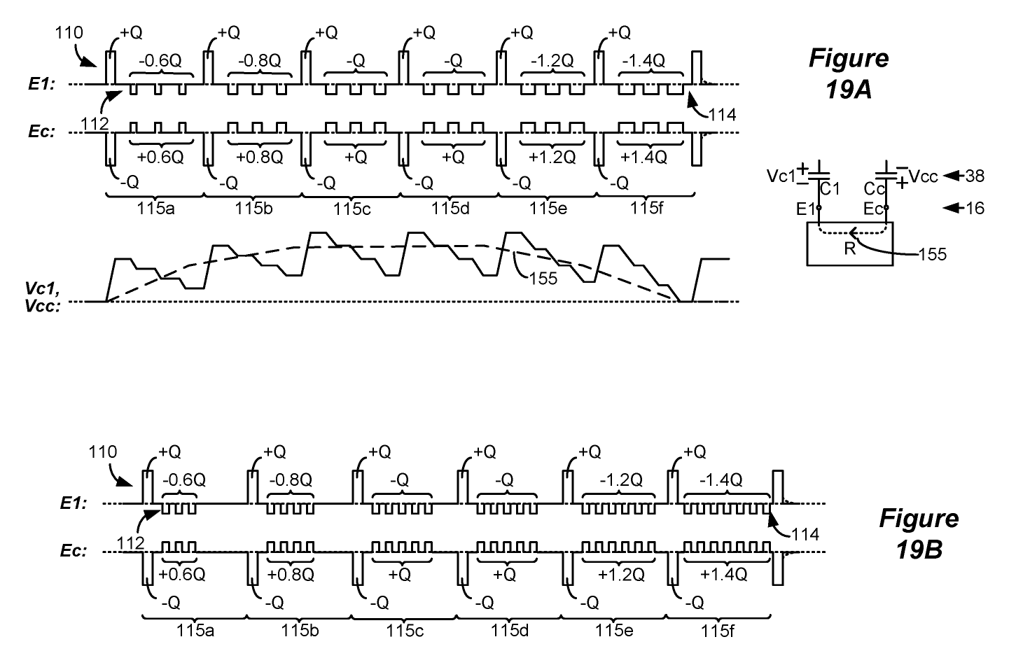

FIGS. 19A and 19B show particular utility to the use of charge imbalanced packets 115. In FIG. 19A, each packet 115 includes an anodic pulse 110 of charge +Q, and three cathodic pulses 112. However, the charge of the cathodic pulses 112 in each packet 115 are varied by adjusting their pulse widths (although as explained earlier, their amplitudes could also be changed).

The cathodic pulses 112 in first packet 115a provide a total charge of -0.6Q, with a net charge in packet 115a of +0.4Q. As shown in the graphs of Vc1 and Vcc, this leaves a charge (voltage) on the DC-blocking capacitors C1 and Cc, and given the polarity of these voltages, a current 155 will flow from Ec to E1, as shown in the circuit diagram. As described in U.S. patent application Ser. No. 16/210,814, filed Dec. 5, 2018, this current 155 comprises a pseudo-constant DC current which can act even during quite periods between the pulses 110 and 112 to assist in the recruitment of neural elements. The cathodic pulses 112 in second packet 115b provide a total charge of −0.8Q, with a net charge in packet 115b of +0.2Q. This increases Vc1 and Vcc further, and increases the current 155 flowing from Ec to E1.

The cathodic pulses 112 in third and fourth packets 115c and 115d provide a total charge of −Q, and so are balanced with the anodic pulses 110. Vc1 and Vcc will not increase further but remain elevated, which allows current 155 to stabilize at a higher level.

The cathodic pulses 112 in fifth and sixth packets 115e and 115f exceed the charge of the anodic pulses 110. Thus, the cathodic pulses 112 in packet 115e have a total charge of −1.2Q, and so packet 115e has a net charge of −0.2Q. This causes Vc1 and Vcc to fall, thus establishing current 155 at a lower level. The cathodic pulses 112 in packet 115f have a total charge of −1.4Q, and so packet 115f has a net charge of −0.4Q. This causes Vc1 and Vcc to fall further to zero, and therefore current 155 is brought back to zero.

Notice that the total net charge between the packets 115a-115f is zero: anodic pulses 110 provide +6Q, while cathodic pulses 112 in sum provide −6Q. Nonetheless, providing charge imbalanced packets can allow a recruitment current 115 to flow. If it is desired to have current flow in the other direction—from electrode E1 to the case electrode Ec, the charge imbalance in each packet simply needs to be modified. That is, packets 115a and 115b can be established to provide a net negative charge, and packets 115e and 115f can be established to provide an offsetting net positive charge.

FIG. 19B shows the same effect as FIG. 19A, but with charge imbalance realized in each packet 115 in a different manner. Specifically, charge imbalance is achieved in FIG. 19B by altering the number of cathodic pulses 112 in each packet. It is assumed in FIG. 19B that each cathodic pulse 112 has a charge at E1 of −0.2Q. Three cathodic pulses 112 are provided in packet 115a, for a total charge of −0.6Q, and a net charge in packet 115a of +0.4Q. Four cathodic pulses 112 are provided in packet 115b, for a total charge of −0.8Q, and a net charge in packet 115b of +0.2Q. Five cathodic pulses 112 are provided in packets 115c and 115d, for a total charge of −Q, and a net charge in packets 115c and 115d of zero. Six cathodic pulses 112 are provided in packet 115e, for a total charge of −1.2Q, and a net charge in packet 115e of −0.2Q. Seven cathodic pulses 112 are provided in packet 115f, for a total charge of −1.4Q, and a net charge in packet 115f of −0.4Q. This operates to the same effect as the pulses in FIG. 19A.

The various waveforms illustrated to this point can be combined in different manners, even if such combinations are not illustrated in the figures. For example, the location of anodic and cathodic pulses 110 and 112 can be switched in a packet (FIG. 7B, 7C); the cathodic pulses can be of different shapes but of the same or different charge (FIGS. 8 and 9); more than one anodic pulse 110 can be used (FIG. 10); more than one lead-based electrode can be used along with the case electrode (FIG. 11); the anodic pulse can be passive (FIG. 12); the case electrode Ec need not be used (FIG. 13); cathodic pulses 150 need not be therapeutic (FIG. 14); the anodic and cathodic pulses 110 and 112 can occur at different frequencies (FIG. 15); a single cathodic pulse 112 can be used with many anodic pulses (FIG. 16); the anodic and cathodic pulses 110 and 112 need not be charge balanced within a packet 115 (FIG. 17); but a plurality of imbalanced packets can in sum be charge balanced (FIGS. 18-19B). It is not practical to illustrate all of these possible combinations, but it should be understood that any combination of these techniques can be used in a practical implementation and are within the scope of this disclosure.

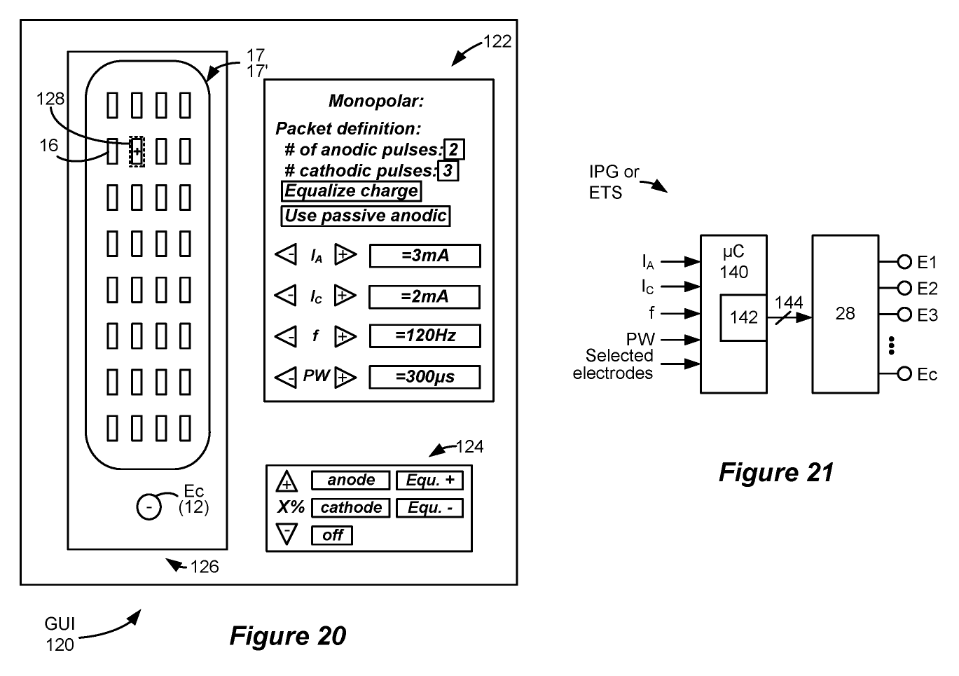

FIG. 20 shows a Graphical User Interface (GUI) 120 which can be used to program an IPG or ETS to provide the waveforms described earlier. GUI 120 may be provided on an external device, such as the external controller 60 or clinician programmer 70 of FIG. 5. One skilled in the art will understand that the particulars of the GUI 120 will depend on where the external device's software is in its execution, which may depend on the GUI selections the clinician or patient has previously made. The instructions for GUI 120 can be stored on a non-transitory computer readable media, such as a solid state, optical, or magnetic memory, and operable within the control circuitry of the relevant external device.

FIG. 20 shows the GUI 120 at a point allowing for the manual setting of stimulation parameters for the patient. A stimulation parameters interface 122 is provided in which specific stimulation parameters can be defined for a stimulation program. In particular, interface 122 may comprise a means for setting monopolar stimulation in which the case electrode Ec is selected as an active electrode, although this isn't required. Adjustable settings for stimulation parameters are shown, including the amplitude I_{A} of the anodic pulses 110 and amplitude I_{C} of the cathodic pulses 112, their frequency f, and their pulse width PW. It is assumed in this example that the pulse widths PW of the anodic and cathodic stimulation pulses 110 and 112 will be the same (e.g., FIG. 7A), although as discussed earlier this isn't necessary, and instead the pulse width of each can be specified in the GUI 120. Means can also be provided for defining the packets 115 of anodic and cathodic pulses 110 and 112, including the number of each. As shown, an option may be provided to allow the charge to be equalized at the selected electrodes and within each packet 115. Note that this may involve automatic computation or adjustment of the other stimulation parameters. For example, if two anodic pulses 110 and three cathodic pulses 112 are specified, the control circuitry of the external device may set the anodic and cathodic currents I_{A} and I_{C} to achieve charge balance within each packet 115 (see FIG. 10). An option may also be provided to allow the anodic pulse 110 to comprise a passive anodic pulse, as described earlier with respect to FIG. 12. If this option is selected, anodic current I_{A} may be set to a “not applicable” value. FIG. 20 is merely one example by which the waveforms described earlier can be defined; more complicated user interface aspects not shown can be provided to allow for the programming of the waveforms in their various alternatives as previously described.

Stimulation parameters relating to the electrodes 16 are made adjustable in an electrode parameter interface 124. Electrodes are manually selectable in a leads interface 126 that displays a graphical representation of the electrode array 17 or 17′ (one or more permanent or trial leads) that has been implanted in a particular patient (a paddle lead 19 is shown as one example). A cursor 128 (or other selection means such as a mouse pointer) can be used to select a particular electrode in the leads interface 126. Buttons in the electrode parameter interface 124 allow the selected electrode (including the case electrode, Ec) to be designated as an anode, a cathode, or off (The case electrode Ec may also be automatically selected if monopolar stimulation is to occur). The electrode parameter interface 124 further allows the amount of the current that each selected electrode will receive to be specified in terms of a percentage, X. This was explained earlier with reference to FIG. 11, which shows how current could be split between electrodes E1 and E2, with each receiving 50%. Note that GUI 120 provides a relatively simple manner for defining the pulses described earlier.

FIG. 21 shows an IPG or ETS capable of forming the pulses as specified by the GUI 120 of the external device. As discussed in the Introduction, the stimulation parameters entered from the GUI 120 can be wirelessly transmitted by the external device 60 or 70 to an antenna in the IPG or ETS, including the anodic and cathodic amplitudes I_{A} and I_{c}, pulse frequency f, pulse width(s) PW, the selected electrodes, etc. The stimulation parameters, once wirelessly received, are provided to control circuitry 140. Control circuitry 140 may comprise a microcontroller for example, such as Part Number MSP430, manufactured by Texas Instruments, which is described in data sheets at http://www.ti.com/lsds/ti/microcontroller/16-bit_msp430/overview.page?DCMP=MCU_other& HQS=msp430. The control circuitry 140 more generally can comprise a microprocessor, Field Programmable Grid Array, Programmable Logic Device, Digital Signal Processor or like devices. Control circuitry 140 may also be based on well-known ARM microcontroller technology. Control circuitry 140 may include a central processing unit capable of executing instructions, with such instructions stored in volatile or non-volatile memory within or associated with the control circuitry. Control circuitry 140 may also include, operate in conjunction with, or be embedded within an Application Specific Integrated Circuit (ASIC), such as described in U.S. Patent Application Publications 2008/0319497, 2012/0095529, 2018/0071513, or 2018/0071520, which are incorporated herein by reference. The control circuitry 140 may comprise an integrated circuit with a monocrystalline substrate, or may comprise any number of such integrated circuits operating as a system. Control circuitry may also be included as part of a System-on-Chip (SoC) or a System-on-Module (SoM) which may incorporate memory devices and other digital interfaces.

In FIG. 21, the control circuitry 140 includes pulse logic 142, which receives the stimulation parameters and forms various control signals 144 for the stimulation circuitry 28. Such control signals 144 specify the timing and polarity of the stimulation pulses appearing at each of the selected electrodes, as well as the amplitude of the current each selected electrode will provide.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A stimulator device, comprising:
a plurality of electrode nodes, each electrode node configured to be coupled to one of a plurality of electrodes configured to contact a patient's tissue; and
stimulation circuitry configured to provide a repeating packet of pulses at at least two of the electrode nodes selected to create a stimulation current through the patient's tissue,
wherein the pulses in each packet at at least one of the at least two electrode nodes comprise at least one monophasic anodic pulse and a plurality of monophasic cathodic pulses, wherein the at least one monophasic anodic pulse comprises a first amplitude, and wherein the plurality of monophasic cathodic pulses each comprise an amplitude less than the first amplitude.

2. The stimulator device of claim 1, wherein the at least one monophasic anodic pulse and the plurality of monophasic cathodic pulses in each packet are separated by gaps during which the stimulation current is not created through the patient's tissue.

3. The stimulator device of claim 1, wherein the amplitudes of the plurality of monophasic cathodic pulses are equal in each packet.

4. The stimulator device of claim 1, wherein each packet comprises only the at least one monophasic anodic pulse and the plurality of monophasic cathodic pulses.

5. The stimulator device of claim 1, wherein the at least one monophasic anodic pulse and the plurality of monophasic cathodic pulses in each packet are charge balanced at each of the at least one electrodes.

6. The stimulator device of claim 1, wherein the at least one monophasic anodic pulse and the plurality of monophasic cathodic pulses in each packet are not charge balanced at each of the at least one electrodes.

7. The stimulator device of claim 1, wherein the pulses in each of the packets comprise a single monophasic anodic pulse and a plurality of monophasic cathodic pulses.

8. The stimulator device of claim 1, wherein each packet further comprises a passive charge recovery phase, wherein during the passive charge recovery phase the stimulation circuitry is configured to short the at least two of the electrode nodes to a reference voltage.

9. The stimulator device of claim 1, wherein the first amplitude of the at least one monophasic anodic pulse and each amplitude of the plurality of monophasic cathodic pulses are selected to be therapeutically effective to recruit neural elements in the patient's tissue.

10. The stimulator device of claim 1, wherein the first amplitude of the at least one monophasic anodic pulse is selected to be therapeutically effective to recruit neural elements in the patient's tissue, but wherein each amplitude of the plurality of monophasic cathodic pulses is selected to not be therapeutically effective to recruit neural elements in the patient's tissue.

11. The stimulator device of claim 1, wherein the at least one monophasic anodic pulse comprises a passive monophasic anodic pulse, wherein the stimulation circuitry is configured to provide the passive monophasic anodic pulse by shorting the at least two of the electrode nodes to a reference voltage.

12. The stimulator device of claim 1, wherein the stimulation circuitry is configured to provide the at least one monophasic anodic pulse and the plurality of monophasic cathodic pulses at a constant frequency at each of the at least two of the electrode nodes.

13. The stimulator device of claim 1, further comprising a case for housing the stimulation circuitry, wherein the case is conductive, and wherein the conductive case comprises one of the plurality of electrodes.

14. The stimulator device of claim 13, wherein one of the at least two of the electrode nodes comprises an electrode node coupled to the conductive case.

15. The stimulator device of claim 14, further comprising at least one implantable lead wherein some of the electrodes are located on the at least one implantable lead.

16. The stimulator device of claim 15, wherein the at least one of the at least two electrode nodes comprises an electrode node coupled to an electrode located on the at least one implantable lead.

17. The stimulator device of claim 1, wherein each electrode node is coupled to an electrode through a DC-blocking capacitor.

18. The stimulator device of claim 1, wherein the first amplitude of the at least one monophasic anodic pulse and each amplitude of the plurality of monophasic cathodic pulses comprise constant current amplitudes.

19. The stimulator device of claim 1, wherein the pulses in each packet at at least one other of the at least two electrode nodes comprise at least one monophasic cathodic pulse corresponding in time to the at least one monophasic anodic pulse and a plurality of monophasic anodic pulses each corresponding in time to one of the plurality of monophasic cathodic pulses, wherein the at least one monophasic cathodic pulse comprises the first amplitude, and wherein each of the plurality of monophasic anodic pulses comprises the amplitude of its corresponding monophasic cathodic pulse.

20. The stimulator device of claim 1, wherein the at least one monophasic anodic pulse and the plurality of monophasic cathodic pulses are charge imbalanced at each of the at least one electrodes in a plurality of the packets, but wherein the charge is balanced at each of the at least one electrodes over a duration of the plurality of the packets.
